(19) United States
(12) Patent Application Publication
(10) Pub. No.: US 2013/0131714 A1
(43) Pub. Date: May 23, 2013

(54) EMBOLIC PROTECTION DEVICE AND METHODS OF MAKING THE SAME

(71) Applicant: Boston Scientific Scimed, Inc., Maple Grove, MN (US)

(72) Inventors: Huisun Wang, Maple Grove, MN (US); James Anderson, Fridley, MN (US); Justin Plessel, Dayton, MN (US)

(73) Assignee: BOSTON SCIENTIFIC SCIMED, INC., Maple Grove, MN (US)

(21) Appl. No.: 13/675,666

(22) Filed: Nov. 13, 2012

Related U.S. Application Data
(60) Provisional application No. 61/559,297, filed on Nov. 14, 2011.

(51) Int. Cl. A61F 2/01

(52) U.S. Cl. CPC A61F 2/01
USPC 606/200

(57) ABSTRACT

An embolic protection device, the device expandable from a first low profile configuration to a second expanded configuration, the device adapted for implantation body lumen, the device comprising an expandable support structure comprising radially expandable tubular first and second end portions and a laterally expandable central portion extending between the first and second end portions.

### CROSS-REFERENCE TO RELATED APPLICATIONS

This application claims priority to U.S. Patent Provisional Application No. 61/559,297 filed Nov. 14, 2011, the entire contents of which are hereby incorporated herein by reference.

### BACKGROUND OF THE INVENTION

The present invention generally relates to embolic protection devices and methods of making and using the same.

Heart disease is a major problem in the United States and throughout the world. Conditions such as atherosclerosis result in blood vessels becoming blocked or narrowed. Aortic valve stenosis (AVS) is a disease of the heart valves in which the opening of the aortic valve is narrowed.

Minimally invasive endovascular aortic arch and valve procedures such as transcatheter aortic valve implantation (TAVI) have become a therapeutic option for patients with severe symptomatic aortic stenosis. TAVI is a procedure that involves implantation of a collapsible prosthetic valve using a catheter-based delivery system. This type of prosthesis can be inserted into the patient through a relatively small incision or vascular access site, and can be implanted on the beating heart without cardiac arrest.

Complications of this procedure include embolization of plaque or thrombus. Embolization can occur from the valve during balloon valvuloplasty and valve deployment or embolization of aortic atheroma can occur during device passage.

Embolizations can be carried downstream to lodge elsewhere in the vascular system. This is particularly problematic in both the left and the right carotid arteries. Such emboli can be extremely dangerous to the patient, capable of causing severe impairment of the circulatory system. Depending on where the embolic material is released, a heart attack or stroke could result, or in the event peripheral circulation is severely compromised, the amputation of a limb may become necessary. Thrombus formation can be particularly problematic in structural heart interventional procedures, particularly in minimally invasive heart valve placement procedure and TAVI procedures.

Cerebral embolism or stroke is the sudden blocking of an artery by a thrombus or clot, or other foreign material which is carried to the site of lodgment via blood flow. Cerebral embolism is one of the major complications of transcatheter structural heart procedures or minimally invasive structural heart procedures.

A number of devices, termed embolic protection devices, have been developed to filter out this debris and reduce the risk of cerebral embolism.

Conventional embolic protection devices are used mainly during the carotid vascular interventional procedure whereas the risk of a thrombus embolism is due to carotid vascular angioplasty or stenting.

There remains a need in the art for an embolic protection device that provides effective protection during a transcatheter aortic valve implantation procedure, but also can be used for an extended protection from thrombus embolism after the procedure.

These and other aspects, embodiments and advantages of the present disclosure will become immediately apparent to those of ordinary skill in the art upon review of the Detailed Description and Claims to follow.

### SUMMARY OF THE INVENTION

In one embodiment, the present invention relates to an embolic protection device, the device expandable from a first low profile configuration to a second expanded configuration, the device adapted for implantation body lumen, the device comprising an expandable support structure comprising radially expandable tubular first and second end portions and a laterally expandable central portion extending between said first and second end portions.

In another embodiment, the present invention relates to an embolic protection device, the device expandable from a first low profile configuration to a second expanded configuration, the device adapted for implantation in a left subclavian artery and brachiocephalic artery and right subclavian artery, and to cover the right and left carotid artery, the device comprising a first end portion configured and arranged for disposition in the left subclavian artery, in the expanded configuration the first end portion is sealingly engageable to a wall of the left subclavian artery, a second end portion configured and arranged for disposition in the brachiocephalic artery and the right subclavian artery, in the expanded configuration the second portion is sealingly engageable to a wall of the right subclavian artery and a middle portion extending between the first end portion and second end portion, in the expanded configuration, the middle portion covers the right and the left carotid artery.

### BRIEF DESCRIPTION OF THE DRAWINGS

FIG. 1 is a perspective side view of one embodiment of an embolic protection device according to the invention in its expanded state.

FIG. 2 is a top view of the device shown in FIG. 1 in its expanded state.

FIG. 3 illustrates a device similar to that shown in FIGS. 1 and 2 expanded in the left and right subclavian arteries and covering the left and right carotid arteries.

FIG. 4 is a side view of a guidewire disposed in the right and left subclavian arteries and through the brachiocephalic and the aortic arch.

FIG. 5 is a side view of a catheter assembly and embolic protection device disposed in the right and left subclavian arteries and through the brachiocephalic and the aortic arch.

FIG. 6 is a side view of an embolic protection device disposed in the right and left subclavian arteries and through the brachiocephalic and the aortic arch. The catheter is being withdrawn from the right subclavian artery.

FIG. 7 is a side view of an embolic protection device disposed in the right and left subclavian arteries and through the brachiocephalic and the aortic arch. The device is disposed on a guidewire.

FIG. 7A is a top down view of the arteries and device as shown in FIG. 7.

FIG. 8 is a side view of an embolic protection device disposed in the right and left subclavian arteries and through the brachiocephalic and the aortic arch wherein the device has been delivered from the left radial artery. The device is shown disposed on a guidewire.

FIG. 9 is a side view of one embodiment of an embolic protection device disposed on a guidewire.

FIG. 9A is an enlarged longitudinal cross-sectional view of an embolic protection device taken at 9A in FIG. 9.

FIG. 10 is a partial side view of the proximal end of one embodiment of an embolic protection device having a recapture mechanism and a corresponding retrieval mechanism on the right side of the figure.

FIG. 11 is a partial side view of the proximal end of one embodiment of an embolic protection device having an alternative recapture mechanism and a corresponding retrieval mechanism on the right side of the figure.

FIG. 12 is a partial side view of the proximal end of one embodiment of an embolic protection device having an alternative recapture mechanism and a corresponding retrieval mechanism on the right side of the figure.

FIG. 13 is a side view of one embodiment of an embolic protection device shown disposed within a delivery device.

FIG. 14 is a side view of one embodiment of an embolic protection device shown disposed on a mandrel.

FIG. 15 is a side view of an alternative embodiment of an embolic protection device having a tapered structure wherein the larger diameter end is configured and arranged for disposal in the brachiocephalic artery and the smaller diameter end is configured and arranged for disposal in the left subclavian artery.

FIG. 15A is a side view illustrating a device similar to that shown in FIG. 15 disposed in the brachiocephalic artery, through the aortic arch and into and the left subclavian artery.

FIG. 16 is a top down view of an alternative embodiment of an embolic protection device.

FIG. 17 is a side view of an embolic protection device similar to that shown in FIG. 16.

FIG. 18 is a top down view of an alternative embodiment of an embolic protection device.

FIG. 19 is a top down view of an alternative embodiment of an embolic protection device.

FIG. 20 is a side view of an alternative embodiment of an embolic protection device.

FIG. 21 is a side perspective view of an alternative embodiment of an embolic protection device including a frame 82 and a membrane 84 disposed on the inner surface of the frame 82. FIG. 18 is a top down view of an alternative embodiment of an embolic protection device.

FIG. 22 is a side perspective view of one embodiment of a mandrel which can be employed to form an embolic protection device which is radially expandable at either end and laterally expandable in the middle.

FIG. 23 is a top down view of a mandrel similar to that shown in FIG. 22.

FIG. 24 is a side perspective view of a mandrel similar to that shown in FIG. 22 having an embolic protection device disposed thereon.

FIG. 25 is a side view of a mandrel similar to that shown in FIGS. 22 and 23 having an embolic protection device disposed thereon.

FIG. 26 illustrates an alternative method and device for forming an embolic protection device, the method and device including shaping dies.

FIG. 27 is a top down view of FIG. 26.

### DETAILED DESCRIPTION

While embodiments of the present disclosure may take many forms, there are described in detail herein specific embodiments of the present disclosure. This description is an exemplification of the principles of the present disclosure and is not intended to limit the disclosure to the particular embodiments illustrated.

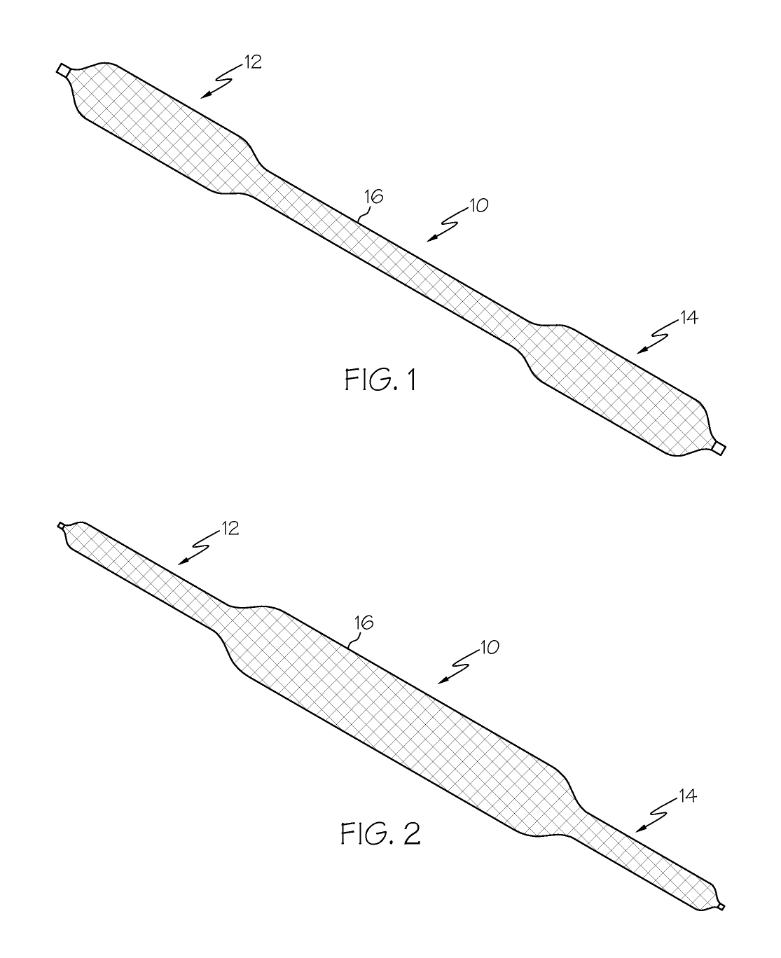

Turning now to the figures, FIG. 1 is a perspective side view illustrating one embodiment of an embolic protection device 10 according to the invention. Device 10 includes radially expandable end portions 12, 14 and a laterally expandable central portion 16. The radially expandable end portions 12, 14 can be clearly seen in their expanded state. FIG. 2 is a top down view illustrating the same device as that shown in FIG. 1 but the laterally expandable central portion 16 can be more clearly seen in its expanded state. Device 10 is closed at either end.

The device is configured and arranged for placement in the aortic arch area and is disposed and deployed in the left subclavian artery and the right subclavian artery of the brachiocephalic artery wherein the central portion 16 of the device 10 covers the left and right carotid arteries for embolic protection.

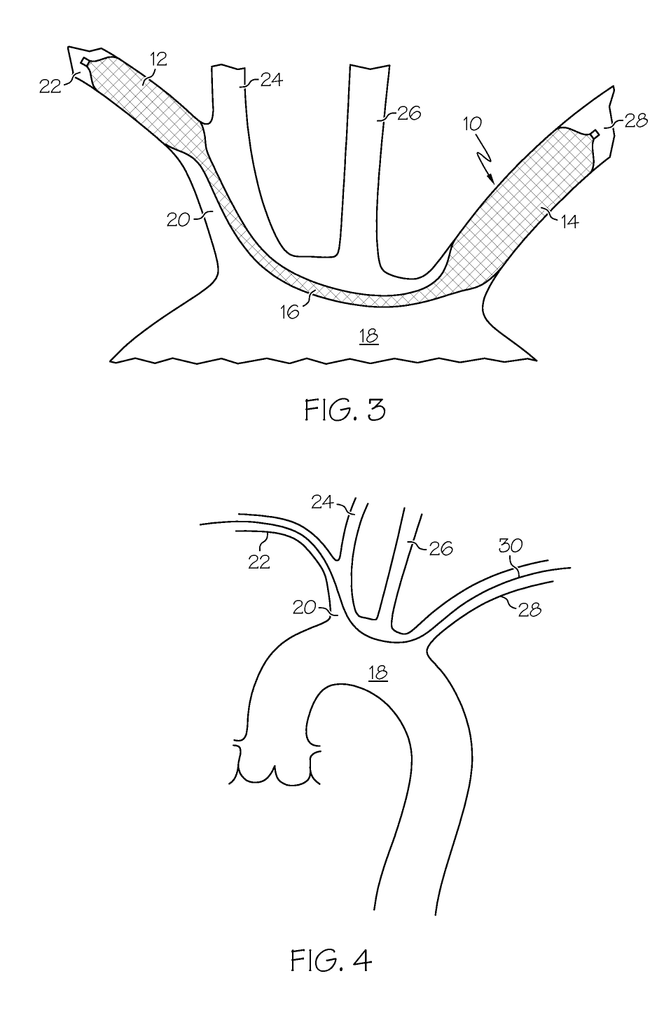

FIG. 3 illustrates device 10 deployed and expanded in a patients vasculature in the aortic arch area 18, namely, end portion 12 of device 10 is radially expanded in the right subclavian artery 20 and engages the wall thereof, end portion 14 of device 10 is radially expanded in the right subclavian artery 22 and engages the wall thereof, and the middle portion 16 of device 10 is expanded and covers the right carotid artery 24 and the left carotid artery 26 and provides protection from emboli that can be generated during structural heart procedures such as placement of an implantable prosthesis in the heart.

The device can be delivered through the vasculature via a catheter delivery device which will be explained in more detail below, via either the left radial artery through the left subclavian artery to the aortic arch or via the right subclavian artery.

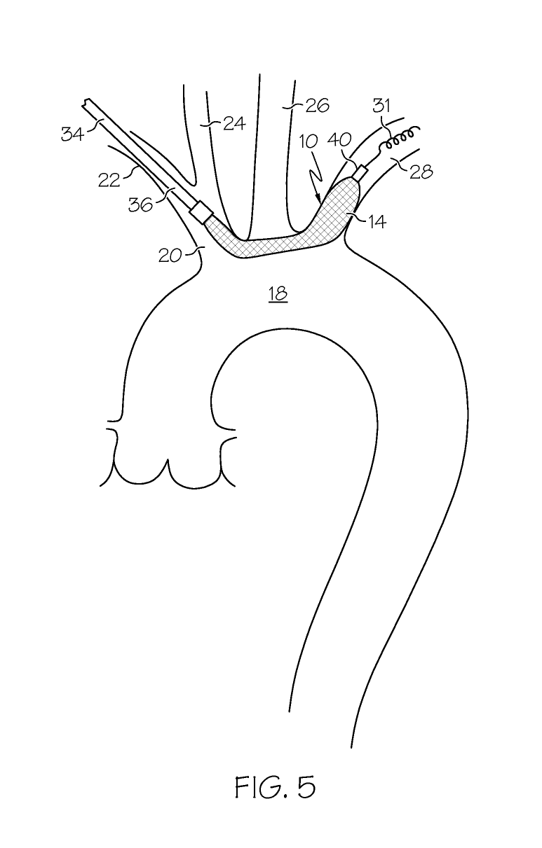

FIGS. 4-7 illustrate one method of delivering the device via the right radial artery into the right subclavian artery 22 passing through the brachiocephalic artery and the aortic arch 18 and finally into the left subclavian artery 28.

A guidewire 30 is first delivered via the left radial artery into the left subclavian artery 28 and advanced through the aortic arch 18 into the brachiocephalic artery 20 and finally into the right subclavian 22.

A delivery catheter 34 comprising a sheath 36 in which device 10 is seated for delivery is then advanced over guidewire 30 from the right radial artery into the right subclavian artery 22 and advanced through the aortic arch 18 into the brachiocephalic artery 20 and finally into the left subclavian artery 28 wherein device 10 can be expanded and deployed. In the embodiments shown in FIGS. 4-7 guidewire 30 has a distal tip that is in the form of a flexible spring coil. An example of a similar guidewire are frontline guidewires available from Boston Scientific and sold under the trademarks of ChoICE®, Luge™, IQ® and Forte®, for example. These guidewires come in diameter sizes of 0.014″, 0.018″ or 0.035″ with a 0.014″ diameter guidewire being most suitable.

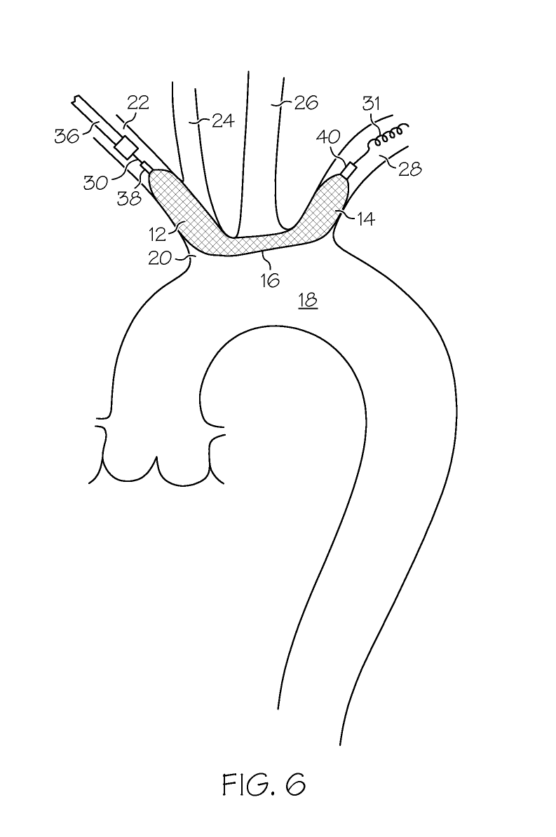

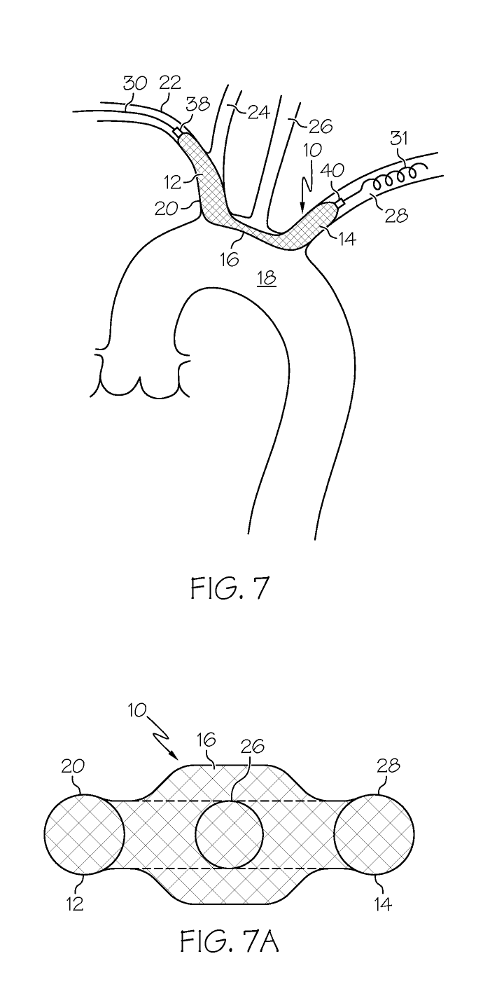

Once in position, sheath 36 can be pulled back to expand the device 10 so that end portions 12, 14 are disposed in the right subclavian artery 22 and the left subclavian artery 28 and the middle portion 16 covers the right carotid artery 24 and left carotid artery 26 as shown in FIG. 7. FIG. 6 illustrates sheath 36 partially pulled back form device 10 wherein end portion 14 of device 10 is shown expanded in the left subclavian artery 28. FIG. 6 illustrates the sheath 36 pulled back completely from device 10 wherein end portion 12 of device 10 is now expanded in the right subclavian artery 22 and middle portion 16 has been laterally expanded in the aortic arch area 18 to cover the right carotid artery 24 and the left carotid artery 26.

FIG. 7A is a top down view taken from FIG. 7 wherein it can be seen that the middle portion 16 of device 10 which is laterally expanded covers the left carotid artery 26 and the radially expanded end portions 12 and 14 can be seen in the brachiocephalic artery 20 and the left subclavian artery 28 respectively.

FIG. 8 illustrates device 10 having been delivered via the left radial artery through the left subclavian artery 30. The distal flexible spring coil 31 of the guide catheter is shown in the right subclavian artery 22 in this case. The process for delivering and deploying the device is in all other respects the same as that discussed with respect to FIGS. 4-7.

Also in the embodiments shown in FIGS. 4-8, device 10 is closed at either end with bands 38, 40 such as radiopaque marker bands.

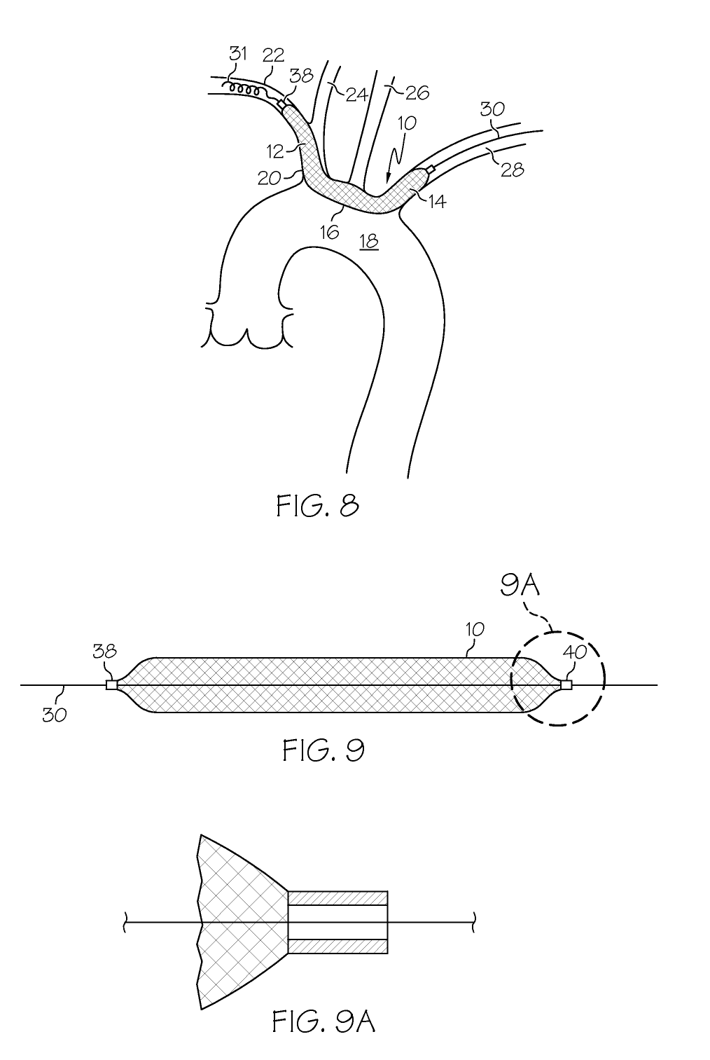

The device 10 can be secured to a guidewire 30 by crimping band 38 onto guidewire 30 as shown in FIG. 9. Band 40 is a hollow ring in which guidewire 30 is slidable therein as shown in FIG. 9A.

The assembly can be constructed such that the guidewire 30 is separate from and slidable within device 10, or device 10 can be fixedly attached to the guidewire 30. In this embodiment, the guidewire 30 is slidable within device 10. The guidwire 30 can be retrieved before device 10 is retrieved.

Bands 38, 40 may be formed from any suitable biocompatible metal or metal alloy. In some embodiments, the bands are formed from a radiopaque metal alloy or radiopaque element loaded polymers. Examples of metals and metal alloys include, but are not limited to, platinum and alloys thereof, gold, silver, tungsten, tantalum, iridium and combinations thereof.

Examples of radiopaque element loaded polymers include, but are not limited to, iodized polycarbonate, barium and bismuth loaded polymers and combinations thereof.

Examples of barium compounds include, for example, barium sulfate.

Examples of bismuth compounds include, but are not limited to, bismuth trioxide, bismuth subcarbonate and bismuth oxychloride.

These lists are intended for illustrative purposes only and not as a limitation on the scope of the present invention. Those of ordinary skill in the art will be aware of alternatives to those materials listed herein.

Device 10 can be employed only during a medical procedure for embolic protection during the procedure, or it can be implanted for a period of time for longer term embolic protection.

Band 38 at the proximal end of the device 10 can be configured and arranged for recapture and retrieval of the device 10 from a patient's body lumen. Examples include, but are not limited to loops, threaded champfer captures, detents or hooks.

The retrieval wire may include the corresponding capture mechanism, for example, hooks, screws, springs or loops.

Moreover, when one or both ends of the device are pulled, the openings in the device will close together more tightly and can trap emboli within the device.

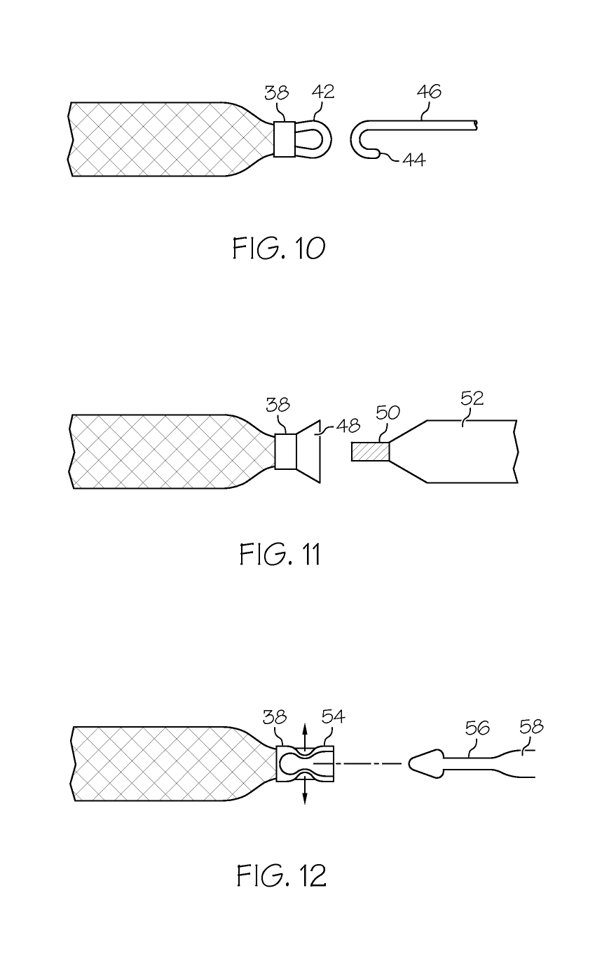

FIG. 10 is a partial side view of the proximal end of device 10 including a band 38 with a loop 42 connected thereto. Also shown in FIG. 10 is the corresponding hook 44 which may be formed integrally with the retrieval wire 46 in the distal end thereof for recapturing device 10. Alternatively, a hook may be attached to the distal end of a wire rather than formed integrally with the wire.

FIG. 11 is a partial side view of the proximal end of device 10 including a band 38 having a threaded champfer capture 48 connected thereto. Also shown in FIG. 11 is the corresponding screw 50 which may either be formed integrally with the retrieval wire 52 or otherwise connected thereto.

FIG. 12 is a partial side view of the proximal end of device 10 including a band 38 having a detent 54 connected thereto. Also shown in FIG. 12 is the corresponding spring 56 which can be formed integrally with the distal end of retrieval wire 58 or otherwise connected thereto.

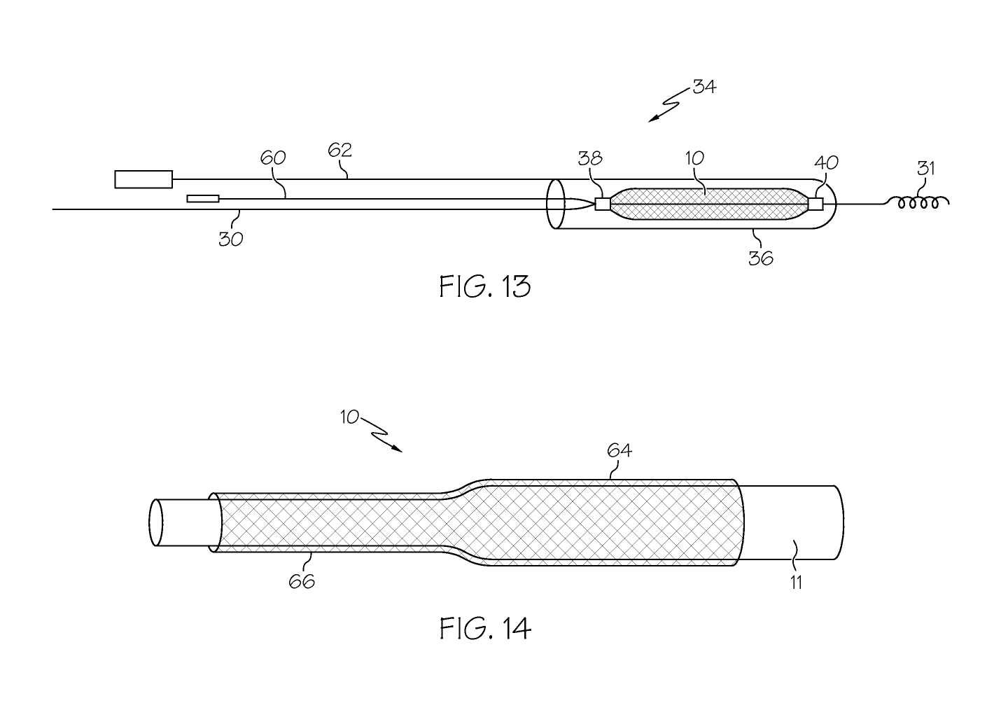

FIG. 13 is a side view of an alternative embodiment of a catheter delivery device 34 which may be employed herein. Catheter delivery device 34 includes a guidewire 30 slidably disposed within device 10 which is disposed in a sheath 36. Catheter delivery device 34 further includes a device control wire or retrieving wire 60 and a proximal shaft 62 which is connected to sheath 36 and is a hollow tubular member. Device 10 is fixedly connected to device control wire 60 at band 38 such as by crimping band 38 onto device control wire 60. Device control wire 60 thus remains with device 10 during the medical procedure and is then employed to remove device 10 once the procedure has been concluded. In this embodiment, device 10 is not implanted in the patient but is only employed for embolic protection and filtering during the medical procedure.

Device 10 can be formed from a variety of materials and with a variety of configurations including, but not limited to, membranes, mesh, braids, weaves, roves, knits, interwinding helical fibers, interconnected serpentine bands, a closed cell stent-like structure and so forth, the material having openings therein that are configured to divert larger emboli and to collect smaller emboli therein. In a mesh pattern, for example, the openings are suitably about 100 microns to about 400 microns.

The openings in the mesh are dynamic from an open device configuration to a closed device configuration. For example, as the device is expanded the openings may be up to about 300 microns and as the device is collapsed and closed, the openings may be as small as about 40 microns so as to capture and remove emboli from the body when the device is withdrawn. These sizes apply to patterns other than mesh as well.

Alternatively, the openings can be smaller so as to divert emboli, for example, during a transcatheter aortic valve implantation (TAVI) procedure.

In some embodiments, the device is formed from a self-expanding material such as a self expanding metal alloy or a self-expanding polymer. In one embodiment, the device is formed from nitinol.

In one embodiment the device has an expanded diameter of about 8-10 mm and a total length of about 4-6 cm.

Various alternative embodiments of device 10 can be employed herein. In one embodiment shown in FIG. 14, device 10 comprises a stepped structure wherein a larger radially expandable end 64 is configured for placement in the brachiocephalic artery and a smaller radially expandable end 66 is configured for placement in the left subclavian artery. Device 10 is shown disposed on a mandrel 11 used for forming device 10. Device 10 can be heat set after formation on the mandrel.

Typical heat set conditions for a device formed from nitinol, for example, may include temperatures in the range of about 490° C. to about 800° C. The time for heat set varies depending on mass, size of the device and fixturing. For a device formed from stainless steel, fixture forming the wire below annealing temperature, for example less than about 425° C. is desirable. Of course these conditions may be changed depending on the material employed for formation of the device.

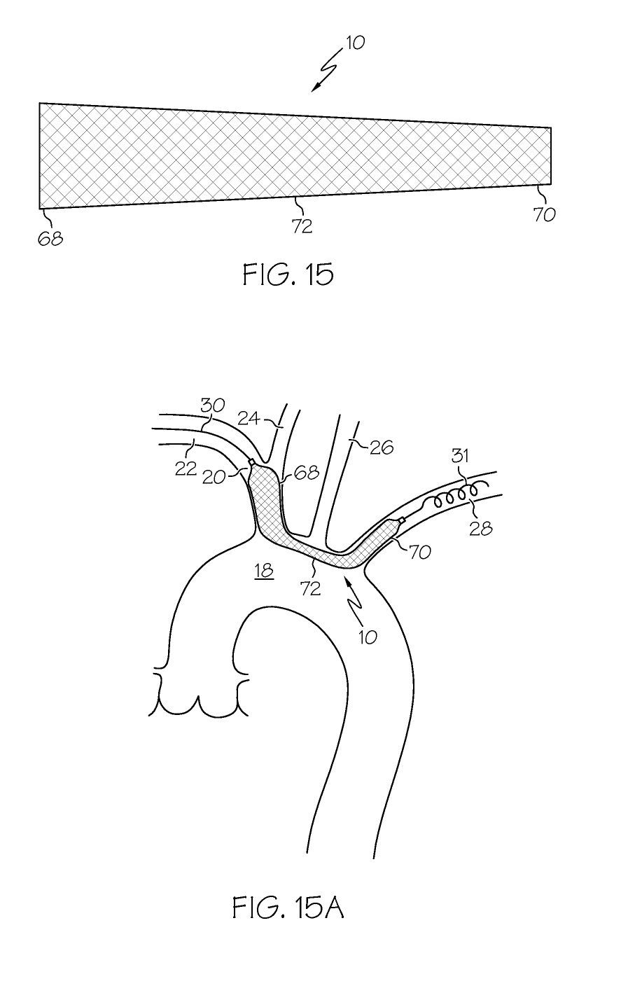

FIG. 15 is an alternative embodiment of device 10 wherein device 10 has a tapered structure with a larger radially expandable end 68 tapering to a smaller radially expandable end 70. FIG. 15A illustrates device 10 disposed in the vasculature wherein end portion 68 is disposed and expanded in the brachiocephalic artery 20 and covers both the right subclavian artery 22 and the right carotid artery 24. End portion 70 of device 10 is disposed and expanded in the left subclavian artery 28 and wherein the middle portion 72 of device 10 covers the left carotid artery 26. Device 10 is closed at either end. Device 10 is shown disposed over a guidewire 30 having a flexible, spring coil at one end. This device is shown delivered via the right subclavian artery 22 but can also be delivered from the left subclavian artery 28 as well.

The end portion 70 for expansion the left subclavian artery 28 suitably has an expanded diameter of about 12 mm while end portion 68 for expansion in the brachiocephalic artery 20 suitably has an expanded diameter of about 14 mm. Delivery diameters are about 1-2 mm for both ends (4-7 Fr, 0.035″-0.080″).

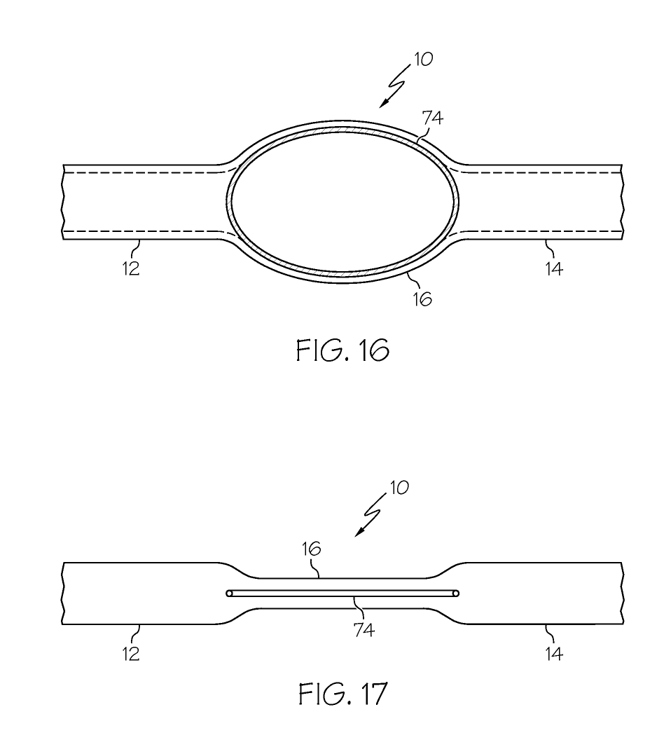

In another embodiment illustrates in FIGS. 16 (top down view) and 17 (side view), a self-expanding ring 74 such as a nitinol ring is placed in a stent-like tube to form the radially expandable middle portion 16. End portions 12, 14 are radially expandable.

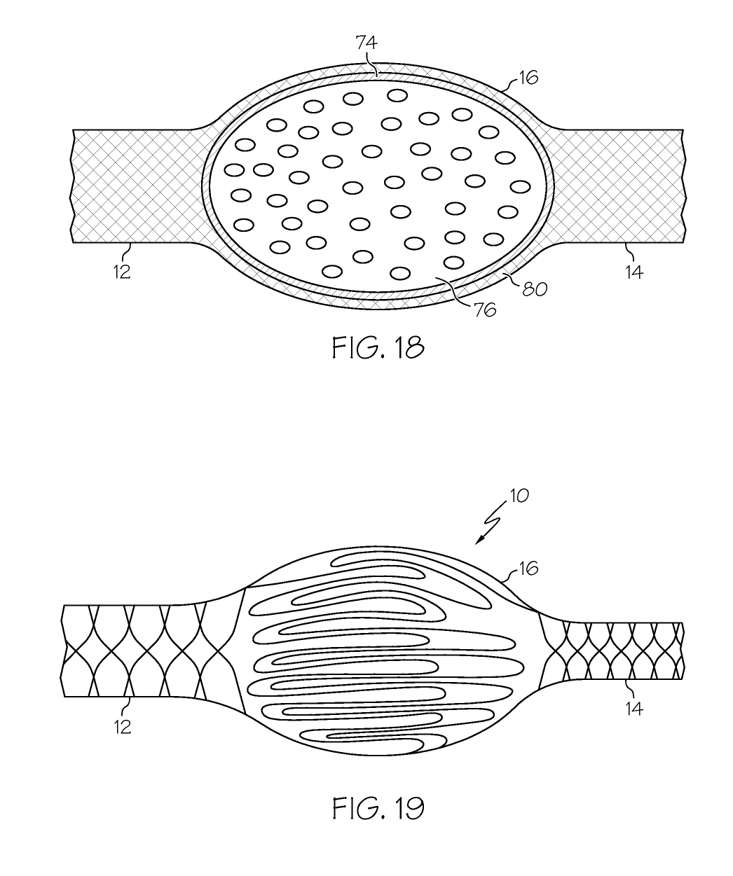

FIG. 18 illustrates an alternative embodiment of a device 10 similar to that shown in FIGS. 16 and 17 wherein the device 10 includes a self-expanding ring 74 having a membrane 76 connected to the frame 80 of the device 10. Ends portions 12, 14 are radially expandable. Membrane 76 can be formed of any suitable biocompatible polymeric material. One example is a polyurethane membrane.

The membrane 76 can be affixed to the device 10 using any suitable method including adhesive bonding using a biocompatible adhesive, or laser or fusion welding.

FIG. 19 illustrates an alternative embodiment wherein the central portion 16 of the device 10 has a different pattern than end portions 12, 14. The central portion 16 is independently expandable laterally or in the aorta plane axis covering the left and right carotid arteries wherein the pattern has an opening size of about 100 to 200 microns and functions to divert emboli from entering the carotid arteries.

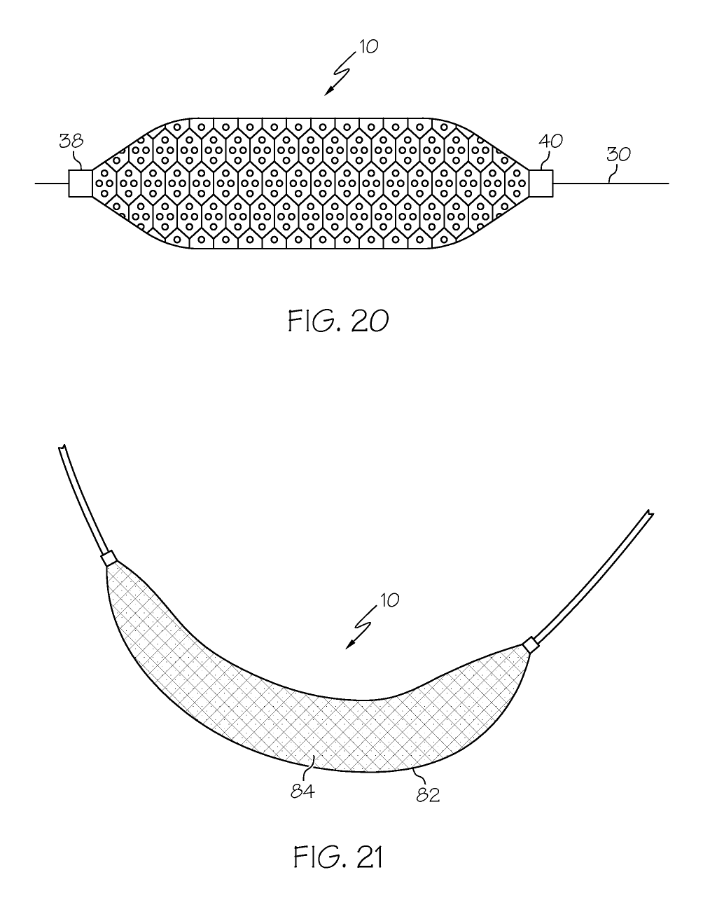

As shown in FIG. 20, device 10 may comprise a closed cell stent-like structure including both small and large elements resembling a honeycomb pattern. The pattern may be cut using any suitable method including laser cutting the pattern into a tubular stent perform as is known in the art. The large elements provide structure while the smaller elements function as a filter to block or deflect emboli. The stent-like structure of the device provides vessel wall apposition, vessel patency and protection from large emboli by diversion, e.g. about 200 microns to about 400 microns when fully expanded, while maintaining the blood flow therethrough. The central portion of the device may be comprised of a nitinol ring, for example, a 0.003-0.006 flat or round nitinol wire, along with a smaller emboli diverting material, for example, a polyurethane membrane having holes sizing of about 100 microns to about 200 microns.

FIG. 21 illustrates an embodiment wherein the stent-like structure includes a membrane 84 on the inner surface of a frame 82. Frame 82 can be formed from any suitable material including metals and metal alloys such as shape memory metal alloys. In one embodiment, the frame is formed from nitinol.

Shape memory polymers may also be employed herein including thermoset and thermoplastic polymers. Examples include, but are not limited to, polyimides, polyether-ether-ketones (PEEK), elastomeric polyurethanes, covalently cross-liked polyurethanes, and so forth.

Membrane 82 may be formed from any suitable porous polymeric material. Examples of suitable materials include, but are not limited to, thermoplastic polymers and thermoplastic elastomeric polymer materials such as polyurethanes, polyether-block-amides and nylons. In one embodiment, the membrane is formed from a polyurethane.

The pores may be provided in the membrane using any suitable method. One example is to employ laser cutting.

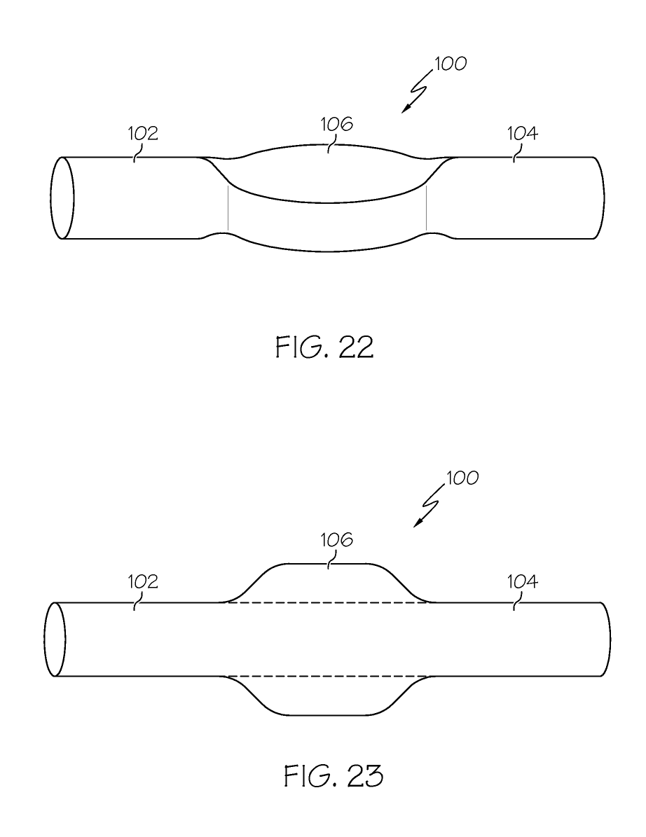

The device 10 can be made using a variety of methods. In one embodiment, device 10 is formed on a shaped mandrel having circular end portions 102, 104 and a flat middle portion 106 as shown in FIG. 22. FIG. 23 is a top down view of mandrel 100.

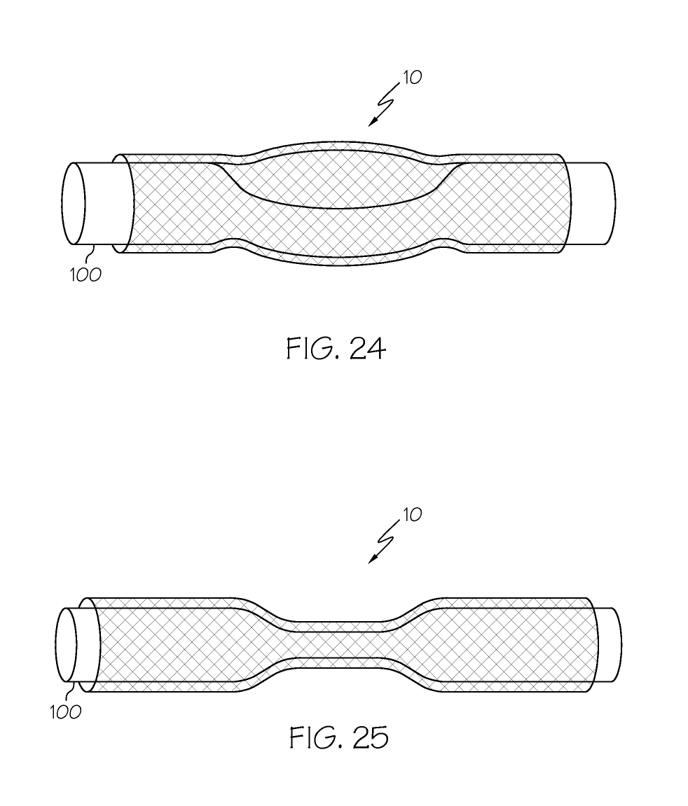

FIGS. 24 (side perspective view) and FIG. 25 (side view) represents device 10 being formed on mandrel 100. In a specific embodiment, a nitinol stent is formed on the shaped mandrel 100 and then heat set for retention of the shape.

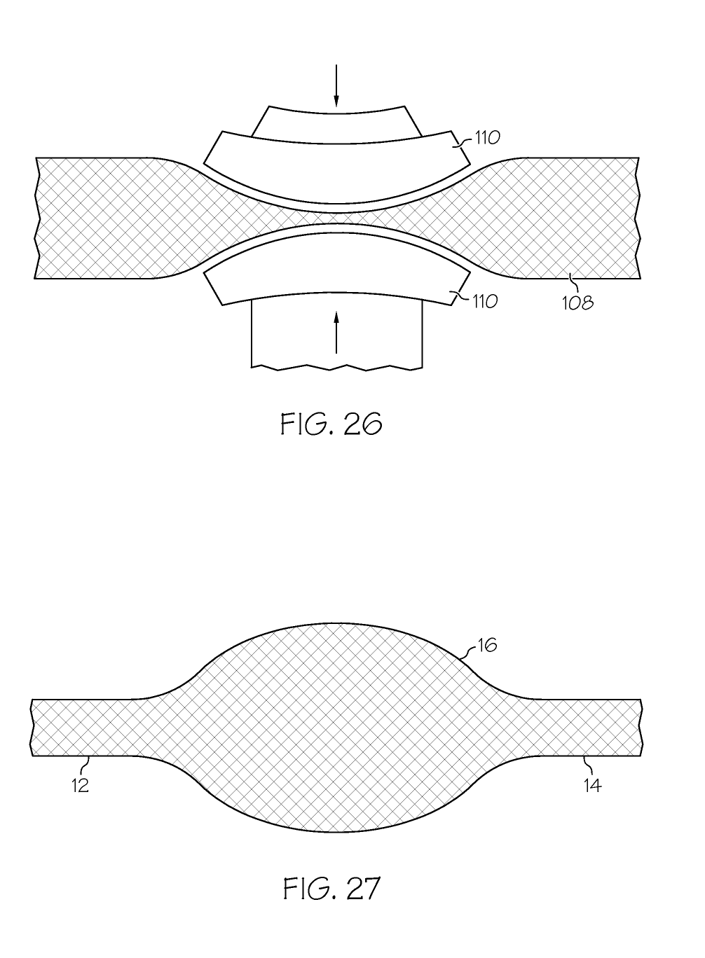

In an alternative embodiment, a die, such as a heat shape die or cold work die is employed to flatten the middle portion of a tubular stent-like structure as shown in FIG. 26. FIG. 27 is a top down view showing tube 108 after shaping with die 110 wherein the central portion 16 and radial end portions 12, 14 of device 10 are formed.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

1. An embolic protection device, the device expandable from a first low profile configuration to a second expanded configuration, the device adapted for implantation body lumen, the device comprising:
an expandable support structure comprising radially expandable tubular first and second end portions; and
a laterally expandable central portion extending between said first and second end portions.

2. The embolic protection device of claim 1, the device comprising a porous structure comprising openings therein, said openings are sized and configured to allow fluid to flow therethrough.

3. The embolic protection device of claim 1 wherein said device is self-expanding.

4. The embolic protection device of claim 3 wherein said device comprises a shape memory metal.

5. The embolic protection device of claim 4 wherein said shape memory metal comprises nitinol.

6. The embolic protection device of claim 1 wherein said support structure comprises a closed cell structure.

7. The embolic protection device of claim 1 wherein said support structure comprises a plurality of interconnected serpentine bands.

8. The embolic protection device of claim 1 comprising a mesh, braid, weave, rove or interwinding helical fibers.

9. The embolic protection device of claim 1 wherein said expandable support structure further comprises a layer of membrane, mesh, weave, rove, braid or interwinding helical fibers.

10. The embolic protection device of claim 9 wherein said layer comprises polyurethane.

11. The embolic protection device of claim 1 wherein said central portion of said expandable support structure further comprises a nitinol ring.

12. The embolic protection device of claim 1 wherein said device comprises a proximal end and a distal end, said device is closed at each of the proximal end and the distal end.

13. The embolic protection device of claim 12 wherein said device is closed at each of the proximal end with a first metallic band and at the distal end with a second metallic band.

14. The embolic protection device of claim 13 wherein each of said metallic bands is a radiopaque marker band.

15. The embolic protection device of claim 13 disposed about a guidewire, the device is crimped onto the guidewire at the proximal end with said metallic band and is slidable in the distal end of the device at said metallic band.

16. The embolic protection device of claim 1 comprising a distal end and a proximal end, the device further comprising at least one recapture mechanism, the recapture mechanism connected to said device at least at one of the proximal end or distal end of said device.

17. The embolic protection device of claim 16 wherein said recapture mechanism comprises a loop, a threaded chamfer capture, a detent or hook.

18. The embolic protection device of claim 17 in combination with a retrieval device, the retrieval device comprising a retrieval mechanism which corresponds to the recapture mechanism of the embolic protection device, capturing means is a hook, screw, spring or loop.

19. The embolic protection device of claim 13, the device further comprising a recapture mechanism, the recapture mechanism is connected to the metallic band at least at the proximal end or distal end of the device.

20. An embolic protection device, the device expandable from a first low profile configuration to a second expanded configuration, the device adapted for implantation in a left subclavian artery and brachiocephalic artery and to cover a right and left carotid artery, the device comprising:
a first end portion configured and arranged for disposition in the left subclavian artery, in the expanded configuration the first end portion is sealingly engageable to a wall of the left subclavian artery;
a second end portion configured and arranged for disposition in the brachiocephalic artery and the right subclavian artery, in the expanded configuration the second portion is sealingly engageable to a wall of the right subclavian artery; and
a middle portion extending between the first end portion and second end portion, in the expanded configuration, the middle portion covers the right and the left carotid artery.
